(19) United States
(12) Patent Application Publication
(10) Pub. No.: US 2011/0144638 A1
(43) Pub. Date: Jun. 16, 2011

(54) Localized Shockwave-Induced Tissue Disruption

(75) Inventors: Tammo Heeren, Aliso Viejo, CA (US); John C. Huculak, Mission Viejo, CA (US); Steven W. Kovalcheck, Aliso Viejo, CA (US)

(73) Assignee: Alcon Research, LTD., Fort Worth, TX (US)

(21) Appl. No.: 12/637,362

(22) Filed: Dec. 14, 2009

(51) Int. Cl. A61F 9/007; A61B 18/18

(52) U.S. Cl. 606/41

(57) ABSTRACT

A high-intensity pulsed-electrical-field (HIPEF) apparatus removes ocular tissue from a localized portion of an eye by delivering one or more focused shockwaves to that tissue. In one embodiment, for example, the apparatus generates one or more electrical pulses, delivers the one or more focused shockwaves to ocular tissue by applying the generated electrical pulses to a HIPEF probe, and then removes the ocular tissue disrupted by the one or more focused shockwaves via aspiration. To mitigate risk of damage to adjacent ocular tissue, the apparatus delivers the one or more focused shockwaves with energy substantially limited to the tissue being removed. The HIPEF apparatus is, therefore, especially advantageous in the context of cataract surgery where cataract tissue need be broken apart and removed without damaging adjacent tissue associated with the lens capsule required to implant an intraocular lens.

### TECHNICAL FIELD

The present invention relates generally to the field of eye surgery and more particularly to methods and apparatus for inducing disruption of ocular tissue within a localized portion of an eye during eye surgery using focused shockwaves.

### BACKGROUND

Techniques and apparatus for dissociation and removal of highly hydrated macroscopic volumes of proteinaceous tissue from the human eye have been previously disclosed. In particular, techniques for dissociation and removal of highly hydrated macroscopic volumes of proteinaceous tissue using rapid variable direction energy field flow fractionization have been disclosed by Steven W. Kovalceck in U.S. patent application Ser. No. 11/608,877, filed 11 Dec. 2006 and titled “System For Dissociation and Removal of Proteinaceous Tissue” (hereinafter “the Kovalcheck application”), the entire contents of which are incorporated herein by reference.

The techniques disclosed in the Kovalcheck application were described in detail in terms of vitreoretinal surgery, for removing ocular tissue such as vitreous tissue. As explained in the Kovalcheck application, prior art procedures have relied for decades on mechanical or traction methods such as: 1) tissue removal with shear cutting probes (utilizing either a reciprocating or rotary cutter); 2) membrane transection using scissors, a blade, or vitreous cutters; 3) membrane peeling with forceps and picks; and 4) membrane separation with forceps and viscous fluids. While improvements in mechanisms, materials, quality, manufacturability, system support, and efficacy have progressed, many of the significant advancements in posterior intraocular surgical outcomes have been primarily attributable to the knowledge, fortitude, skill, and dexterity of the operating ophthalmic physicians.

Rather than using such classical mechanical means, the Kovalcheck application disclosed using a high-intensity pulsed electric field (HIPEF) to engage, decompose, and remove ocular tissues. The Kovalcheck application was based on the discovery that a transient change in tissue condition caused by the application of a HIPEF is satisfactory for removal of ocular tissues such as vitreous tissue. That is, vitreous tissue need not be obliterated or disrupted on a molecular level to be removed—rather, momentary dissociation of proteinaceous complexes is all that is needed for removal.

In some contexts, however, such momentary dissociation may be insufficient or otherwise undesirable for removing ocular tissues. During cataract surgery, for example, a surgeon must remove almost the entire natural lens of an eye, including cataract tissue, and replace the lens with an intraocular lens implant. Removal of cataract tissue often requires the surgeon to break apart or chop the tissue into smaller pieces, i.e., a process more disruptive to the tissue than a mere momentary dissociation thereof.

### SUMMARY

Embodiments of the present invention remove ocular tissue from a localized portion of an eye during eye surgery by delivering one or more focused shockwaves to that tissue. As the focused shockwaves propagate to the ocular tissue, the tissue is mechanically disrupted with sufficient force to break apart or chop the tissue into smaller pieces for removal via aspiration. To mitigate risk of damage to adjacent tissue, the one or more focused shockwaves have energy substantially limited to the tissue being removed.

More particularly, a high-intensity pulsed electrical field (HIPEF) apparatus includes a high voltage pulse generator, a HIPEF probe, and an aspiration system. The high voltage pulse generator generates one or more electrical pulses. The HIPEF probe then delivers one or more focused shockwaves to ocular tissue within a portion of the eye by applying the generated electrical pulses to an electrode of the probe. The aspiration system removes ocular tissue that is disrupted by the one or more focused shockwaves.

In some embodiments, the HIPEF apparatus is configured to deliver focused shockwaves by forming and collapsing cavitation bubbles adjacent to the ocular tissue to be removed. In other embodiments, the HIPEF apparatus is configured to deliver focused shockwaves by temporarily displacing the HIPEF probe or the ocular tissue.

With the above described advantages, the present invention is particularly well suited in the context of cataract surgery. For example, the present invention may selectively remove cataract tissue from a natural lens of the eye by delivering one or more focused shockwaves to that tissue. As the one or more focused shockwaves are delivered with energy substantially limited to the cataract tissue being removed, the risk of damage to adjacent tissue (e.g., tissue associated with the lens capsule required to implant an intraocular lens) is mitigated.

Of course, those skilled in the art will appreciate that the present invention is not limited to the above features, advantages, contexts or examples, and will recognize additional features and advantages upon reading the following detailed description and upon viewing the accompanying drawings.

### BRIEF DESCRIPTION OF THE DRAWINGS

FIG. 1 is a perspective view of an exemplary high-intensity pulsed electric field (HIPEF) probe used for eye surgery.

FIG. 2 is an enlarged perspective view of the tip of the probe of FIG. 1.

FIG. 3 is a schematic diagram of a high-intensity pulsed electric field (HIPEF) apparatus according to some embodiments of the invention.

FIGS. 4A-4C illustrate various embodiments for delivering one or more focused shockwaves to cataract tissue within a natural lens of an eye.

FIG. 5 is a logic flow diagram illustrating one embodiment of a method for removing ocular tissue from a localized portion of an eye during eye surgery.

### DETAILED DESCRIPTION

The present disclosure describes an apparatus and method for removing ocular tissue from a localized portion of an eye during eye surgery using one or more shockwaves. In the context of cataract surgery, for example, the apparatus and method remove cataract tissue from the natural lens of an eye using one or more shockwaves. The apparatus and method deliver the one or more shockwaves to the tissue being removed with energy that is substantially focused on that tissue, thereby mitigating risk of damage to adjacent ocular tissue.

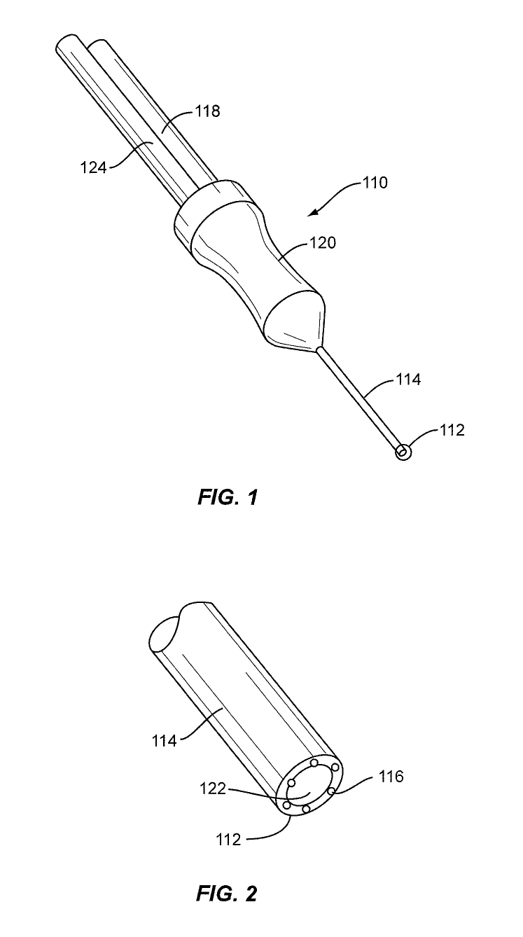

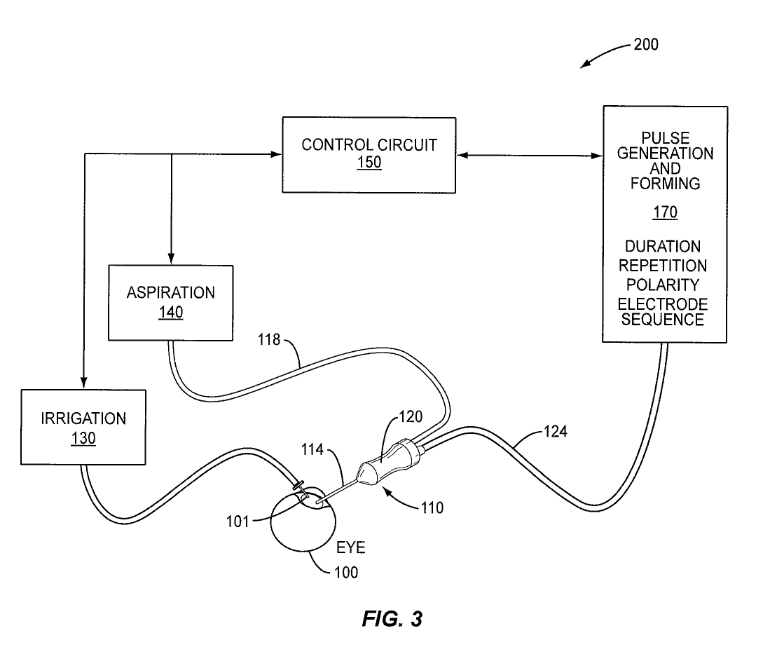

More particularly, one or more focused shockwaves are delivered using a high-intensity pulsed electric field (HIPEF) probe 110 shown in FIG. 1. The HIPEF probe 110 may be similar to that described in the Kovalcheck application (U.S. patent application Ser. No. 11/608,877), but is modified for removing other types of ocular tissue by alternatively or additionally delivering one or more focused shockwaves as described below. Regardless, the HIPEF probe 110 comprises a hollow probe needle 114 extending from a handle 120 to a probe needle tip 112, as well as an aspiration line 118 and a transmission line 124. FIG. 2 illustrates details of the probe needle 114 and probe needle tip 112. As shown in FIG. 2, at least one electrode 116 is exposed at the tip 112 and surrounds an aspiration lumen 122. The at least one electrode 116 is connected to the transmission line 124 for applying one or more generated electrical pulses and delivering the one or more focused shockwaves. The aspiration lumen 122 is connected to the aspiration line 118 for providing an aspiration pathway for ocular tissue disrupted by the one or more focused shockwaves.

FIG. 3 illustrates additional operational details for delivering one or more focused shockwaves to cataract tissue with a HIPEF apparatus 200, which includes the HIPEF probe 110. Using handle 120, the tip 112 of the probe 110 may be inserted by a surgeon into the natural lens 101 of an eye 100. Using standard visualization processes, cataract tissue is engaged by the tip 112 at the distal end of the hollow probe 114. An irrigation system 130 of the apparatus 200 may be activated by control circuit 150. The irrigation system 130 delivers irrigation fluid to the cataract tissue in order to control the electrical impedance of the cataract tissue. Meanwhile, a high voltage pulse generator 170 of the apparatus 200 (which includes a pulse-forming network and switching circuit, in some embodiments) generates one or more electrical pulses. The HIPEF probe 110 then delivers one or more focused shockwaves to cataract tissue within the natural lens 101 by applying the generated electrical pulses to the at least one electrode 116 at the tip 112, via the transmission line 124. The HIPEF probe 110 delivers the one or more focused shockwaves with energy that dissipates quickly with distance from the HIPEF probe 110. Accordingly, the one or more focused shockwaves have energy substantially limited to the cataract tissue being removed, mitigating damage to adjacent tissue (e.g., tissue of the lens capsule necessary to permit implantation of an intraocular lens). As the one or more focused shockwaves propagate to the cataract tissue, the tissue is mechanically disrupted with sufficient force to break apart or chop the tissue into smaller pieces. The disrupted cataract tissue is then removed from the natural lens 101 and drawn through the aspiration lumen 122 and aspiration line 118 by an aspiration system 140 e.g., to a collection module.

Those skilled in the art will readily appreciate that the present invention is not limited by the specific manner in which the HIPEF apparatus 200 delivers the one or more focused shockwaves using the HIPEF. Indeed, those skilled in the art will understand that such focused shockwaves can be delivered using a variety of techniques.

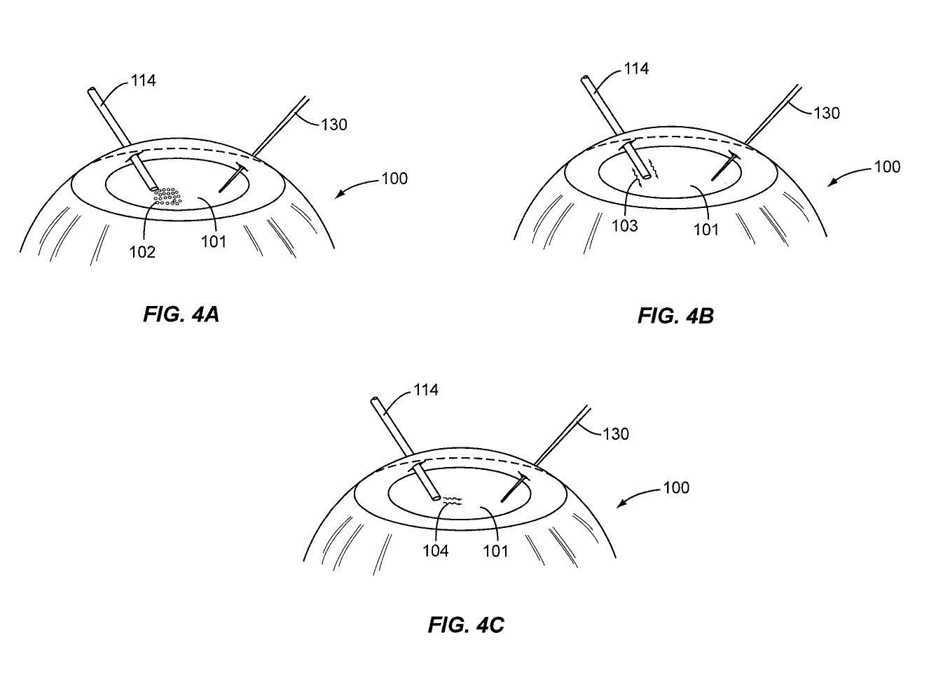

In the embodiment of FIG. 4A, for example, the HIPEF apparatus 200 is configured to deliver one or more focused shockwaves to cataract tissue by forming and collapsing cavitation bubbles adjacent that tissue. Specifically, the probe needle 114 and irrigation system 130 are inserted into the eye 100, to engage the natural lens 101. The HIPEF apparatus 200 is then configured to form cavitation bubbles 102 adjacent cataract tissue to be removed, e.g., by surrounding the tissue with irrigation fluid and delivering a first shockwave to the tissue. After the cavitation bubbles 102 have been formed, the HIPEF apparatus 200 is configured to force the collapse of those bubbles 102, e.g., by delivering a second shockwave to the tissue. The timing of the delivered shockwaves may be adjusted to control the intensity of the bubble collapse. Regardless, the collapse of the cavitation bubbles 102 disrupts the cataract tissue with sufficient force to break apart or chop the tissue into smaller pieces. The aspiration system 140 then removes the cataract tissue disrupted by the bubble collapse. This process may be repeated a number of times until all of the cataract tissue within the natural lens 101 has been broken apart and removed by the HIPEF apparatus 200.

In another embodiment, illustrated in FIG. 4B, the HIPEF apparatus 200 is configured to deliver one or more focused shockwaves to cataract tissue by temporarily displacing the HIPEF probe 110. More particularly, the electrical pulses applied to the electrode(s) 116 are generated with a shape, rate, length, and other pulse parameters that cause the HIPEF probe 110 to be temporarily displaced, e.g. toward the cataract tissue (denoted by line 103 in FIG. 4B). The temporary displacement of the HIPEF probe 110 in turn causes an abrupt, nearly discontinuous change in the applied HIPEF, thereby delivering one or more focused shockwaves to the cataract tissue. Similar to the above embodiment, the one or more focused shockwaves disrupt and otherwise break apart the cataract tissue. This process may be repeated over multiple time intervals, with the parameters of the electrical pulses alternating over those intervals, such that the HIPEF probe 110 is temporarily displaced in alternating directions. Once all of the cataract tissue within the natural lens 101 has been broken apart, the aspiration system 140 removes the disrupted cataract tissue.

In yet another embodiment, illustrated in FIG. 4C, the HIPEF apparatus 200 is configured to deliver one or more focused shockwaves to cataract tissue by inducing displacement of cataract tissue. Specifically, the HIPEF apparatus 200 applies the generated electrical pulses to the electrode(s) 116 to create a HIPEF. The apparatus 200 then induces displacement of cataract tissue (denoted by line 104 in FIG. 4C) within the natural lens 101 by applying the HIPEF to that tissue. This causes an abrupt, nearly discontinuous change in the medium through which the HIPEF propagates, thereby delivering one or more focused shockwaves to the cataract tissue. These shockwaves disrupt the cataract tissue, which is removed by the aspiration system 140.

Although the technique taught herein has been described above in the context of disrupting and removing cataract tissue, those of ordinary skill in the art will understand the applicability of the disclosed invention for disrupting and removing other types of ocular tissues as well. Generally, therefore, the particular ocular tissue to which the disclosed invention is directed does not limit the invention.

Indeed, regardless of the specific manner in which the HIPEF apparatus 200 delivers the one or more focused shockwaves, the pulse generation circuit 170 generates the pulse shape, the pulse repetition rate, the pulse train length, and other parameters of the electrical pulses based on delivering a shockwave with certain desired characteristics. That is, disruption of different types of ocular tissues may be optimal with shockwaves that have different intensities, durations, and/or other characteristics. Accordingly, electrical pulse parameters for delivering a shockwave with characteristics optimal for disrupting a specific ocular tissue may be pre-configured in the HIPEF apparatus 200, whereby a surgeon may select between different pre-configurations based on the type of ocular tissue being removed by the apparatus 200.

Moreover, FIGS. 1-4 above have illustrated the irrigation system 130 as being configured to deliver the irrigation fluid by way of cannula independent from the HIPEF probe 110. However, those of ordinary skill in the art will understand that the irrigation system 130 may additionally or alternatively be configured to deliver the irrigation fluid through one or more irrigation channels within the probe 110.

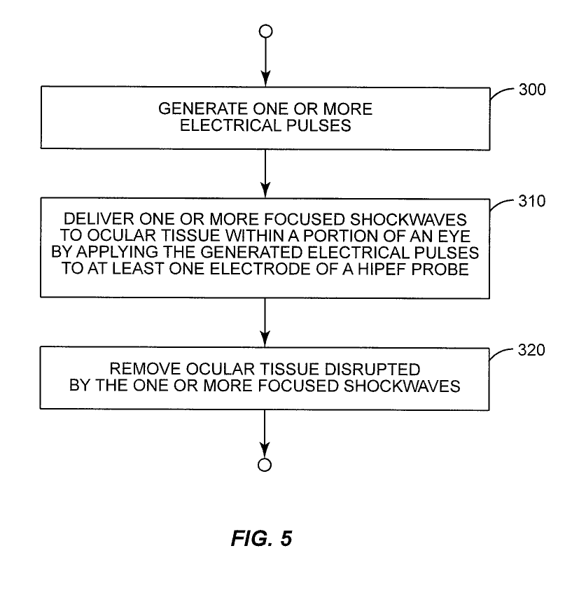

Accordingly, those of ordinary skill in the art will readily appreciate that the HIPEF apparatus 200 generally performs the method illustrated in FIG. 5 for removing ocular tissue from a localized portion of an eye 100 during eye surgery. As shown in FIG. 5, the pulse generation circuit 170 generates one or more electrical pulses (Block 300). The HIPEF probe 110 then delivers one or more focused shockwaves to ocular tissue within a portion of an eye 100 by applying the generated electrical pulses to at least one electrode 116 of the probe 110 (Block 310). The aspiration system 140 then removes ocular tissue disrupted by the shockwaves (Block 320).

Of course, this embodiment and all of the other embodiments described above for removing ocular tissue from a localized portion of an eye during eye surgery were given for purposes of illustration and example. Those skilled in the art will appreciate, therefore, that the present invention may be carried out in other ways than those specifically set forth herein without departing from essential characteristics of the invention. The present embodiments are thus to be considered in all respects as illustrative and not restrictive, and all changes coming within the meaning and equivalency range of the appended claims are intended to be embraced therein.

What is claimed is:

1. A method for removing ocular tissue from a localized portion of an eye during eye surgery, the method comprising:
generating one or more electrical pulses;
delivering one or more focused shockwaves to ocular tissue within a portion of an eye by applying the generated electrical pulses to at least one electrode of a high-intensity pulsed-electrical-field (HIPEF) probe; and
removing from said portion of the eye ocular tissue disrupted by said one or more focused shockwaves.

2. The method of claim 1, wherein said ocular tissue comprises cataract tissue.

3. The method of claim 1, wherein delivering one or more focused shockwaves comprises forming and collapsing cavitation bubbles adjacent said ocular tissue by applying the generated electrical pulses to said at least one electrode of the HIPEF probe.

4. The method of claim 1, wherein delivering one or more focused shockwaves comprises applying the generated electrical pulses to said at least one electrode of the HIPEF probe and temporarily displacing the HIPEF probe.

5. The method of claim 1, wherein delivering one or more focused shockwaves comprises:
applying the generated electrical pulses to said at least one electrode of the HIPEF probe to create a HIPEF; and
inducing displacement of ocular tissue within said portion of the eye by applying the HIPEF to that ocular tissue.

6. The method of claim 1, wherein removing from said portion of the eye ocular tissue disrupted by said one or more focused shockwaves comprises removing the ocular tissue by aspiration.

7. A high-intensity pulsed-electrical-field (HIPEF) apparatus for removing ocular tissue from a localized portion of an eye during eye surgery, the HIPEF apparatus comprising:
a pulse generation circuit configured to generate one or more electrical pulses;
a HIPEF probe comprising at least one electrode and configured to deliver one or more focused shockwaves to ocular issue within a portion of an eye by applying the generated electrical pulses to said at least one electrode; and
an aspiration system configured to remove from said portion of the eye ocular tissue disrupted by said one or more focused shockwaves.

8. The HIPEF apparatus of claim 7, wherein said ocular tissue comprises cataract tissue.

9. The HIPEF apparatus of claim 7, wherein the HIPEF probe is configured to deliver one or more focused shockwaves by forming and collapsing cavitation bubbles adjacent said ocular tissue, by applying the generated electrical pulses to said at least one electrode of the HIPEF probe.

10. The HIPEF apparatus of claim 7, wherein the HIPEF probe is configured to deliver one or more focused shockwaves by applying the generated electrical pulses to said at least one electrode of the HIPEF probe and temporarily displacing the HIPEF probe.

11. The HIPEF apparatus of claim 7, wherein the HIPEF probe is configured to deliver one or more focused shockwaves by:
applying the generated electrical pulses to said at least one electrode of the HIPEF probe to create a HIPEF; and
inducing displacement of ocular tissue within said portion of the eye by applying the HIPEF to that ocular tissue.
